(19) United States
(12) Patent Application Publication
(10) Pub. No.: US 2018/0235730 A1
(43) Pub. Date: Aug. 23, 2018

(54) ARRANGEMENT, DEVICE AND METHOD FOR PRODUCING AN ORTHODONTIC APPARATUS AND DEVICE FOR INDIRECT ADHESION OF AN ORTHODONTIC APPARATUS

(71) Applicant: SONNENBERG CONSULTING AG, Mohlin (CH)

(72) Inventor: Cepand Djamchidi, Kleinmachnow (DE)

(21) Appl. No.: 15/961,368

(22) Filed: Apr. 24, 2018

Related U.S. Application Data
(62) Division of application No. 14/898,852, filed on Dec. 16, 2015.

(30) Foreign Application Priority Data
Jun. 17, 2013 (DE) 102013010186.7

(51) Int. Cl. A61C 7/00; A61B 6/03; A61C 7/20; A61C 7/08; A61C 7/14

(52) U.S. Cl. CPC A61C 7/002; A61B 6/032; A61C 7/20; B33Y 50/00; A61C 7/08; A61C 7/146

(57) ABSTRACT

The invention relates to an instruction for producing all orthodontic apparatuses and/or orthodontic aids which are required during the entire duration of an orthodontic treatment, wherein the arrangement comprises a device for digitally recording a tooth region to be treated orthodontically, a data processing unit and at least one 3D printer unit, wherein the data processing unit controls the at least one 3D printer unit and is or can be connected to the device for digital recording of a tooth region to be treated orthodontically. The invention further relates to a device for producing all orthodontic apparatuses and/or orthodontic aids, in particular a rapid prototyping printer. The invention further relates to a method for producing all orthodontic apparatuses and/or orthodontic aids which are required during the entire duration of an orthodontic treatment. The invention further relates to a device for indirect adhesion of orthodontic apparatuses.

### BACKGROUND

Orthodontic treatment relate to the moving of malpositioned teeth to the desired positions in the oral cavity. Such treatments are carried out for cosmetic reasons, in order to correct for example overbites or highly misaligned teeth. Furthermore, the function of the jaw can also be improved by an orthodontic treatment, if for example better occlusion during chewing is intended to be achieved.

For orthodontic treatment, use is made for example of what are referred to colloquially as fixed braces. Here, brackets are fixed to a patient's teeth so that an arch wire can be placed in the slotted region of each bracket. This arch wire forms a track for guiding the movement of the teeth to a desired location. The ends of these arch wires are usually bent behind a patient's molars.

Also known are what are referred to as “removable braces”. These are distinguished by the fact that they are not fixed in the patient's jaw but can be removed individually by the user. Also known are what are referred to as aligners. Aligner treatment, as it is known, is an orthodontic treatment method for the largely invisible treatment of minor tooth misalignments, which works with a plurality of individually produced, thin, transparent plastics material splints. With the aid of a special computer graphics method, starting from the actual state of the row of teeth, which is recorded in jaw models, a previously determined treatment aim is represented three-dimensionally and subdivided into individual treatment phases. For each of these phases, the separate individual splints are produced industrially, these splints each only being worn for a few weeks. During this time, the teeth are continuously moved in the previously defined direction by targeted application of pressure.

When use is made of known bracket systems, the problem often occurs of defective bonding. Here, the brackets are not stuck to the teeth exactly at the ideal intended positions, and so, under certain circumstances, the desired treatment result does not occur. Accordingly, the prior art discloses the possibility of what is referred to as the indirect sticking of the brackets with a transfer tray. On the basis of an impression of the rows of teeth, a plaster model of the teeth is first of all produced. Then, the tooth model is used to produce a transfer tray which represents a negative of the tooth model. This tooth model is appropriately fitted beforehand with brackets, and so the tray is applied, together with the brackets present therein, to the patient's teeth, with the result that the brackets can be applied to the teeth in one step and in a correct position. According to the conventional production method, the production of the tray is very complicated, since impressions, models etc. have to be produced.

If the orthodontic treatment also provides for the use of aligners, further production processes have to follow in this connection, and so a large number of impressions, production steps and corresponding calculations need to be performed for an orthodontic treatment over a relatively long period.

### SUMMARY

The invention relates to an arrangement for producing any orthodontic appliance and/or orthodontic aid which is required for the entire duration of an orthodontic treatment. Moreover, the invention relates to a device for producing any orthodontic appliance which is required for the entire duration of an orthodontic treatment, the device comprising especially at least one 3D printer unit. Furthermore, the invention relates to a method for producing any orthodontic appliance which is required for the entire duration of an orthodontic treatment. In addition, the invention relates to a device for the indirect sticking of fixed orthodontic appliances, especially a tray.

### BRIEF DESCRIPTION OF THE DRAWINGS

The invention is explained in more detail in the following text by way of an exemplary embodiment with reference to the appended drawings, in which:

FIG. 1 shows an arrangement according to the invention for producing any orthodontic appliance which is required for the entire duration of an orthodontic treatment;

FIG. 2 shows a device according to the invention for producing orthodontic appliances, which comprises a 3D printer, specifically a rapid prototyping printer;

FIG. 3 shows a flowchart relating to a method according to the invention for producing any orthodontic appliance;

FIG. 4 shows an expanded flowchart relating to the method according to the invention for producing any orthodontic appliance;

FIG. 5 shows a further expanded flowchart of the method according to the invention for producing orthodontic appliances; and

FIG. 6 shows a device according to the invention for the indirect sticking of fixed braces.

### DETAILED DESCRIPTION

The aim of the present invention is to specify an arrangement and a device for producing any orthodontic appliance which is required for the entire duration of an orthodontic treatment, i.e. even for a number of years. It is another aim of the present invention to specify an improved method for producing any orthodontic appliance which is required for the entire duration of an orthodontic treatment, i.e. even for a number of years. The method is intended to be simplified such that first of all it requires fewer steps in order to produce orthodontic appliances. Furthermore, the novel method is intended to make it possible for even dentists or orthodontists to be capable of producing orthodontic appliances, thereby dispensing with the involvement of a dental technician and/or of an external laboratory and/or of an external production site.

A further aim of the present invention is to specify a refined device for the indirect sticking of orthodontic appliances, especially a tray.

According to the invention, this aim is addressed by the subject matter of claim 1 with regard to the arrangement, by the subject matter of claim 5 with regard to the device, by the subject matter of claim 12 with regard to the method, and by the subject matter of claim 20 with regard to the device for the indirect sticking of orthodontic appliances.

With regard to the arrangement, the invention is first of all based on the idea of specifying an arrangement for producing any orthodontic appliance and/or orthodontic aid which is required for the entire duration of an orthodontic treatment. According to the invention, this arrangement has a device for digitally capturing a tooth region to be treated orthodontically, a data processing unit and at least one 3D printer or 3D printer unit. The data processing unit controls the at least one 3D printer unit, the data processing unit being connected or connectable to the device for digitally capturing a tooth region to be treated orthodontically.

It should first of all be noted that the tooth region to be treated orthodontically is the entire jaw, i.e. can be the tooth region in the upper jaw and in the lower jaw. Furthermore, it is also possible to use the provided arrangement to produce orthodontic appliances which serve only to treat individual teeth.

The 3D printer or 3D printer unit can preferably be a rapid prototyping printer and/or a CAD/CAM printer. Furthermore, the 3D printer unit can also be understood as being a 3D production unit which comprises for example a milling unit. In the following text, the invention is explained by way of a rapid prototyping printer, it being understood that this is merely an example of a 3D printer.

The data processing unit is a computer with associated software. This computer or the data processing unit is connected or connectable to the rapid prototyping printer(s) and controls the latter.

The orthodontic appliances and/or orthodontic aids can be for example devices for the indirect sticking of orthodontic appliances, for example a tray or a transfer tray. Furthermore, they can be aligners or removable braces or bite splints. In principle, this arrangement is suitable for the production of any orthodontic appliance.

The device for digitally capturing a tooth region to be treated orthodontically may be an intraoral scanner and/or an x-ray device and/or a computed tomograph and/or a digital volume tomograph. The digital capturing of a tooth region to be treated orthodontically or the three-dimensional optical recording can thus take place by means of an intraoral dental camera system which creates a three-dimensional optical recording of the teeth to be treated by means of a triangulation method. Furthermore, it is possible to prepare a three-dimensional x-ray recording of the tooth region to be treated. Such a three-dimensional x-ray recording of the upper jaw or of the lower jaw or of the entire jaw or of the jaw joints or of the jaw joint can be created for example by means of computed tomography (CT) or what is referred to as digital volume tomography (DVT).

In a further embodiment of the invention, it is conceivable for determined x-ray data and determined scan data to be overlaid such that greater precision can be achieved with regard to the diagnostic method.

In digital volume tomography, in order to calculate three-dimensional structures, two-dimensional images are produced as a dataset. A digital volume tomograph consists of a rotating x-ray source and a CCB sensor, which are rotated through 180° or 360° about the patient in a fixed position. Cross sections in all spatial planes and three-dimensional views can be calculated by means of a digital volume tomograph.

Since the device for digitally capturing a tooth region to be treated orthodontically is connected or connectable to the data processing unit, the data captured by the device are sent to the data processing unit. It should be noted that this connecting of the device for digitally capturing a tooth region to the data processing unit can also take place via what are referred to as wireless connection options such as WLAN, Bluetooth or infrared interface.

The data processing unit can furthermore be connected to a data input device or is connectable to such a data input device. In other words, the computer is connected for example to a keyboard and/or what is referred to as a mouse, so that personnel, for example a dentist, or an orthodontist or a tooth technician, can view the data from the data processing unit or amend or supplement said data. The data input device can furthermore be a touchscreen, which is especially easy to clean and disinfect. Input via further terminals such as cell phones and/or smartphones and/or what are referred to as tablets is likewise possible. In this case, it is especially advantageous to make a corresponding input application (app) available on the terminals just mentioned. The data processing unit can form for example, together with a screen, a keyboard and a mouse, a planning unit with the aid of which the orthodontic treatment can be planned. To this end, the data processing unit has a corresponding planning program which makes it possible to use a three-dimensionally generated model of the teeth to be treated or of the tooth region to be treated orthodontically to plan the orthodontic treatment. This planning can be modified individually by operating personnel, i.e. by the dentist, orthodontist, or by a tooth technician. The planning incorporates inter alia the individual treatment steps and/or the selection of treatment instruments.

First of all, an end result or the end position of each individual tooth is defined. The overall orthodontic treatment, which can last for several years, is subdivided into individual steps or individual intermediate results. These individual intermediate results or treatment steps are correlated with corresponding orthodontic appliances, i.e. the optimum orthodontic appliance and/or the optimum orthodontic aid for the particular treatment step is/are filtered out or selected from a multiplicity of orthodontic appliances. It is furthermore possible to prepare additional digital images of the tooth region to be treated orthodontically during ongoing treatment, so that corrections and/or changes to the orthodontic appliances/aids can be carried out. It is advantageous to store the digital images, since the course of treatment is documented.

Subsequently, the particular orthodontic appliance is calculated and virtually constructed, so that datasets relating to the orthodontic appliance to be produced can be used in order to control the rapid prototyping printer so that the particular orthodontic appliance can be printed or produced with the aid of a rapid prototyping printer.

According to an additional independent aspect, the invention is based on the idea of specifying a device for producing any orthodontic appliance which is required for the entire duration of an orthodontic treatment, especially a 3D printer, preferably a rapid prototyping printer.

According to the invention, the 3D printer or rapid prototyping printer has at least one chamber for filling with brackets. Provision is thus made of a storage chamber which stores what are referred to as brackets. Preferably, a plurality of such chambers are provided, so that different bracket sizes or different bracket types and brackets from different manufacturers are stored or present in a number of mutually independent chambers.

Expediently, provision is made for the rapid prototyping printer to comprise at least one chamber for filling with wire. In other words, provision is made of a chamber in which a wire is present. This wire is used for example to produce arch wires which are known in connection with fixed braces. During a treatment phase, a number of different copies of such arch wires, which can all be produced by way of the described arrangement or the device according to the invention, are also necessary.

Furthermore, it is possible for the arrangement to have at least one chamber with prefabricated arch wires, which can be removed for example with the aid of a gripping arm. Preferably, the arrangement identifies all the parts to be removed, such as brackets, arch wires or other aids, with the aid of an RFID code and/or QR code. If such codes are intended to be used, the arrangement preferably has an optical system for identifying the codes.

The chambers containing wire or brackets are provided in order to be able to automatically provide for example a transfer tray with brackets and/or an arch wire.

Furthermore, provision can be made for the device for producing any orthodontic appliance to comprise an automated inventory control system, this inventory control system being connected to a monitoring device for checking the filling levels of the at least one chamber filled with brackets and/or of the at least one chamber filled with wire and/or of the at least one chamber provided with auxiliary elements, or the inventory control system having such a monitoring device. Moreover, it is possible to monitor the plastics materials used by the printer. In a further embodiment of the device according to the invention, a breakdown of the materials to be used for the entire duration of a course of treatment or during individual substeps can be established for a patient with the aid of the inventory control system.

In order for example to be able to provide a transfer tray with brackets and wires, the rapid prototyping printer preferably has a robotic system having at least one gripping device. This robotic system provides the automatic fitting of the trays with brackets. The gripping device grasps for example a bracket from a chamber or storage chamber and subsequently positions the bracket at an intended point on the transfer tray. Expediently, a plurality of gripping devices are provided.

The gripping device can move for example on a rail or rollers in order to reach the produced parts in a better manner. Moreover, it is possible as a result to be able to deposit the produced parts at a different position in the device for further processing. If desired, it is also possible for a plurality of gripping devices to be provided. Furthermore, it is possible to remove the produced parts manually from the 3D printer unit and to deposit them in the working region of the gripping arm.

In a further embodiment of the invention, the rapid prototyping printer can comprise at least one wire bending device. Such a wire bending device is provided so as to be able to already bend the arch wire of a fixed brace in the rapid prototyping printer, so that the fitting of the brackets with the arch wire or the connecting of the brackets to the arch wire can already take place at the stage of production of the transfer tray. The use of ready-made arch wires is also possible with the device according to the invention.

In order to protect the orthodontic appliances produced by means of the rapid prototyping printer, the rapid prototyping printer preferably has a UV-light resistant cover. Any brackets present which are produced directly from plastics material or are coated with a plastics material are also preferably protected from light or UV light in a storage chamber. In this regard, the corresponding storage chamber has a UV-light resistant cover.

Furthermore, provision can be made for the rapid prototyping printer to have a packaging device. Accordingly, finished orthodontic appliances can be automatically packaged. Although provision is made for the orthodontic appliances to be able to be produced in medical practices, so that the orthodontic appliances can be produced directly prior to the insertion thereof into the patient's dentition, if all the orthodontic appliances for the entire duration of an orthodontic treatment are produced in one step or at one time, or the orthodontic appliances are not produced in the medical practice but at a company or a dental technician's, it is advantageous to package the produced orthodontic appliances for reasons of hygiene. Moreover, on account of the packaging of the orthodontic appliances, it is possible to store the latter for the period of time between the production of the appliance and the inclusion thereof in other work steps.

In a further aspect of the invention, provision is made of a method for producing any orthodontic appliance which is required for the entire duration of an orthodontic treatment.

According to the invention, the following method steps are carried out:

Capturing of a tooth region to be treated orthodontically, this being, as already mentioned, either individual teeth or only the lower jaw or upper jaw or the entire jaw. The capturing of a tooth region to be treated orthodontically can take place either with the aid of an impression to be prepared and a model, to be created from the impression, of the tooth region to be treated, or by capturing with the aid of a device for digitally capturing a tooth region to be treated orthodontically. The advantage of the method according to the invention is thus first of all that a plastic tooth model is not necessary. If such a tooth model already exists, however, or is possibly produced for better visualization, this can likewise be used according to the present method. Accordingly, the capturing of a tooth region to be treated orthodontically takes place by way of a plastic tooth model.

Preferably, the capturing of a tooth region to be treated orthodontically should take place with the aid of such a device, since the relinquishment of an impression to be prepared and the model to be produced on the basis of the impression is dispensed with and furthermore corresponding material is saved. The capturing of the tooth region can, in a further development of the invention, be carried out with reference points or reference elements fastened to the patient or to the teeth.

In a further step of the method, a digital three-dimensional model of a tooth region to be treated orthodontically is prepared. If a device for digitally capturing the tooth region to be treated is used, the preparation of a digital three-dimensional model takes place directly following scanning in or the preparation of x-ray images or digital x-ray images (DVT) and the corresponding calculation of the 3D model.

Subsequently, automatic and/or manual creation of a model of a modified tooth arrangement is carried out. In this regard, corresponding planning units can be used with a data processing unit having a corresponding planning program and/or a keyboard and/or a mouse and/or a screen. In other words, when the model of a modified tooth arrangement is created automatically, the planning program undertakes the entire creation. In the case of manual creation, an operator can specify and/or calculate corresponding modified tooth arrangements with the aid of the keyboard and/or a mouse. It is furthermore possible to couple the automatic creation with the manual creation of a model of a modified tooth arrangement, i.e. modifications can be made manually to the automatically created model.

Provided that a model of a modified tooth arrangement is finally present, the preparation of a dataset with regard to the orthodontic treatment steps to be carried out individually in succession takes place. Accordingly, starting from the actual state of the teeth to be treated or of the tooth region to be treated, a comparison with the result to be achieved or the end position of the teeth to be treated a calculation is carried out which individual treatment steps are necessary in order to be able to move the teeth from the actual situation into the end position or into the defined modified tooth position. X-raying and/or scanning is important to predict the movement of the teeth. Therefore, treatment errors and damage to the teeth or to the jaw can be avoided.

Subsequently, linkage of each orthodontic treatment step to be carried out individually in succession with an orthodontic appliance from a selection of a plurality of orthodontic appliances is carried out. Accordingly, the orthodontic appliance which can carry out the respective individual orthodontic treatment steps or is suitable for the respective treatment step is automatically and/or manually defined. For example, it is possible to define in this context that the first step is carried out with what is referred to as a removable brace. Subsequently, a fixed brace becomes necessary. With regard to the fixed brace, the positions of the brackets should be defined. Moreover, in this regard, the dimensions or the geometry of the individual arch wires of the fixed brace should be determined in temporal succession, i.e. individual arch sequences. Finally, provision can be made for what are referred to as aligners to carry out the final corrections, and so the shape and dimensions of the aligners to be used individually in succession should likewise be calculated or defined. All of the mentioned linkages of the individual treatment steps with the orthodontic appliances and the dimensions of the appliances or the data from the respective orthodontic appliances can be stored and assigned to the respective patient files. It is of course possible to vary the listed treatment steps. Provision can also be made to scan and/or x-ray again in order to carry out possible corrections.

Once the linkage of each orthodontic treatment step to be carried out individually in succession with an orthodontic appliance from a selection of a plurality of orthodontic appliances has been carried out, automatic and/or manual creation of a digital model of the selected orthodontic appliance should be carried out. In the case of automatic creation of the digital model, this is carried out via what is referred to as a planning program. In the case of manual creation, this creation is carried out by corresponding operating personnel, and in the case of this manual creation, the corresponding work should be carried out in connection with the data processing unit, i.e. on a computer.

Preferably, a skeletonization process, on the basis of which the selected orthodontic appliance is digitally reduced to a minimum with regard to excess material, is carried out. In what is referred to as a skeletonization process, the contact areas or minimum thicknesses which have at least to be provided with regard to the material of the orthodontic appliance are determined in order to be able to carry out corresponding treatment steps. With the aid of a skeletonization process, material is saved. In addition, a comfortable wearing feel or high wearing comfort is created for the patient, and thus greater acceptance by the patient.

Furthermore, it is possible for a gripping device or robotic system associated with the printing device to be able to insert prefabricated parts and/or elements into the appliance during the printing operation. The elements can be for example screws or wires.

Finally, the provision of a digital dataset of a digital model of the selected and optionally skeletonized orthodontic appliance is carried out. Accordingly, the digital model is converted into a digital dataset. These digital datasets can be stored in a patient file, just like the provided datasets relating to the orthodontic treatment steps to be carried out individually in succession.

What is referred to as a rapid prototyping printer is controlled on the basis of the digital dataset such that the plastics material constituents of the orthodontic appliance can be printed. The plastics material constituents can have any desired colors and patterns.

As already mentioned, the orthodontic appliances can be devices for the indirect sticking of orthodontic appliances, especially trays and/or aligners and/or removable braces. After the plastics material constituents of the orthodontic appliance have been printed, automatic and/or manual fitting of the printed orthodontic appliance with at least one bracket and/or at least one bent wire and/or screws and/or other elements is carried out. This fitting of an orthodontic appliance with brackets and/or a wire is necessary for example in the production of a transfer tray. If automatic fitting is selected, the rapid prototyping printer has a corresponding robotic system and corresponding storage chambers with respect to the brackets and with respect to the wire. Optionally, the rapid prototyping printer can have a wire bending device which can produce the arch wires.

Preferably, automatic capturing of the quantity of brackets and/or wire and/or used and/or required plastics material present in the chambers of a rapid prototyping printer and/or of screws or other elements present in the storage chambers is carried out. The captured data relating to the available quantities of brackets and/or available wire and/or of plastics material are transmitted to an inventory control system. Preferably, it is possible to generate automatic ordering operations with respect to required brackets and/or required wire and/or required plastics material and/or required auxiliary elements, for example screws, with the aid of the inventory control system.

According to a further aspect, the invention is based on the idea of specifying a device for the indirect sticking of orthodontic appliances, especially a tray, this tray or the device for indirect sticking having at least one predetermined breaking point. With the structure of the tray, or with the dimensions of the tray, the gingival line is for the first time not included in the structure.

Once the indirect sticking of for example brackets has been carried out, such that the brackets initially present in the tray and/or the wire that is present are stuck to the tooth region to be treated orthodontically, problems often arise when it comes to removing the transfer tray from the already firmly stuck or fixed brackets and/or the firmly stuck or fastened wire. Accordingly, the tray preferably has at least one predetermined breaking point for when a plurality of brackets are set/locked simultaneously, thereby making it easier to remove the transfer tray. Furthermore, it is possible to configure the tray with the aid of undercuts such that it can additionally be made easier to remove the tray.

In a further embodiment of the invention, the tray is a skeletonized orthodontic appliance which has been reduced to a minimum with regard to excess material.

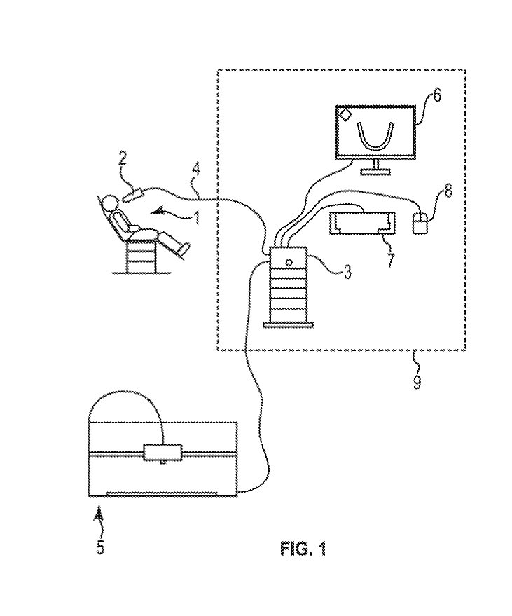

As illustrated in FIG. 1, the arrangement according to the invention for producing any orthodontic appliance which is required for the entire duration of an orthodontic treatment can be located in the rooms of a dental practice or of an orthodontist or of a dental laboratory.

Accordingly, a patient 1 is present in a dentist's chair, while the tooth regions to be treated orthodontically or the entire jaw are captured with the aid of a device for digitally capturing the regions or the jaw. In the present exemplary embodiment, the device for digital capturing is an intraoral scanner 2. This intraoral scanner is connected or connectable to a data processing unit 3. In the present case, a data line 4 is provided, it also being possible to establish the connection by means of Bluetooth or infrared or with the aid of a WLAN connection.

The arrangement according to the invention furthermore comprises a rapid prototyping printer 5, this rapid prototyping printer 5 being controlled by the data processing unit 3. It is possible to couple a plurality of printers together. In the present case, the data processing unit 3 forms a planning unit 9 together with a corresponding planning program, a screen 6, a keyboard 7 and a mouse 8.

According to the exemplary embodiment in FIG. 1, a device for the indirect sticking of orthodontic appliances, specifically a tray, is produced. In general, it is also conceivable for only the individual cap regarding individual teeth to be produced with the aid of the arrangement according to the invention. This relates to all embodiments and to all embodiments of the invention that are illustrated in the following figures.

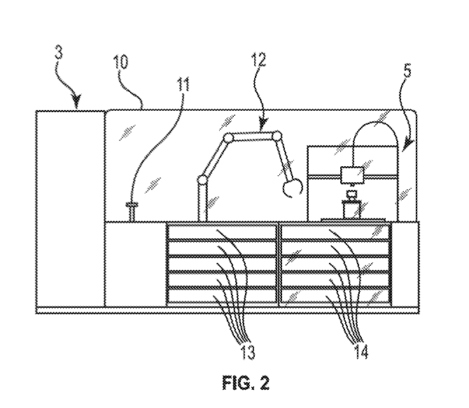

The device according to the invention for producing any orthodontic appliance, which comprises a 3D printer, especially a rapid prototyping printer, is illustrated in detail in FIG. 2.

The illustrated device should be considered as a unit which comprises at least one rapid prototyping printer 5. Also provided is a data processing unit 3, which, in the exemplary embodiment according to FIG. 2, is structurally connected to the further subassemblies. Starting from the data processing unit 3, the data relating to the orthodontic appliance to be produced are sent to the 3D printer 5. The plastics material constituents of the orthodontic appliance are produced for example by means of the rapid prototyping method.

Subsequently, the produced plastics material constituents are transported to a holder 11 for example with the aid of a gripping arm 12 of the 3D printer unit. In this holder 11, the plastics material constituent of the orthodontic appliance is provided for example with further elements and/or aids. In this case, these can be brackets and/or arch wires and/or other aids. The brackets and/or arch wires and/or other aids to be fitted are stored in storage containers or chambers 13. Provision can be made to store a plurality of bracket types and/or bracket sizes. Moreover, provision can be made to store a plurality of wires with different wire diameters.

With the aid of the gripping arm 12, the brackets and/or arch wires and/or aids are placed on the plastics material constituent of the orthodontic appliance and optionally fastened.

Once the orthodontic appliance has been completed, it can be transported from the holder 11 into one of the storage chambers 14 with the aid of the gripping arm 12. Optionally, the illustrated device can have a packaging unit which packages the finished appliances for example in plastics material films and/or containers.

The device is furthermore provided with a cover 10. This is preferably an orange-colored sheet which protects the orthodontic appliances from incident light, especially UV light. The storage chambers 14 are preferably likewise provided with such a sheet. This can also be an additionally attached UV sheet.

It should be noted that all of the components illustrated in FIG. 2 can be connected structurally together. Furthermore, it is conceivable for only individual components, for example the rapid prototyping printing unit 5, the holder 11 and the gripping arm 12 to be able to be connected firmly together. Furthermore it is conceivable for the individual components to merely be able to be arranged alongside one another and to be displaceable in a variable manner.

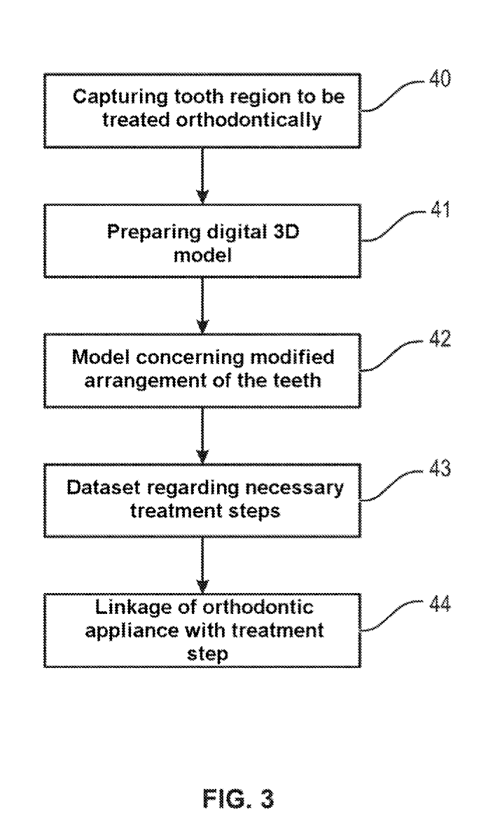

FIG. 3 shows a flow diagram relating to the method according to the invention for producing orthodontic appliances.

According to a first method step 40, a tooth region to be treated orthodontically is captured. For example, a digital three-dimensional model of a tooth region to be treated orthodontically is prepared by means of an intraoral scanner directly on the patient, on a model or alternatively from an impression. This step is indicated by the reference sign 41. The creation of the dataset can furthermore be created on the basis of 3D/DVT images and a scanner image jointly, in order that the movement of the tooth roots is rendered visible. In this way, it is possible to check whether the planned movements are possible and the teeth are not damaged during the orthodontic treatment.

Subsequently, a model of a modified tooth arrangement is created automatically and/or manually. In other words, a virtual preferably three-dimensional model relating to the final position of each tooth to be treated is created. This creation 42 can take place either automatically with a corresponding processing program or manually. The automatic creation takes place for example with the aid of a planning program, the software or the program being able to provide suggestions relating to the end position of the teeth. However, manual processing of the modified tooth arrangement automatically proposed and created is also intended to be possible. In this connection, the dentist or orthodontist can make corresponding amendments to the automatically generated proposal in the planning program by means of the keyboard and/or the mouse and/or a touchscreen and/or a tablet PC.

In a further step 43, a dataset relating to the orthodontic treatment steps to be carried out individually in succession is prepared. Accordingly, a comparison of the actual state with the model of a modified teeth arrangement is carried out. The planning software or the user can subdivide the positional changes of the teeth that are to be carried out into individual treatment steps to be carried out in succession.

Subsequently, each individual treatment step to be carried out in succession can be linked with an orthodontic appliance from a selection of a plurality of orthodontic appliances. This step 44 can in turn be carried out automatically and/or manually, i.e., using a planning program, the operator can make for example corresponding amendments to automatic proposals. In the event of possibly critical planned movements, an automatic piece of advice can be provided, so that the planning of the orthodontic treatment can be considered and corrected.

In step 44, a plurality of different orthodontic appliances can be considered to be suitable for the various treatment steps. The selection of the respective orthodontic appliances can be stored for example in a patient file.

After the orthodontic appliances to be used individually in succession have been defined 44, automatic and/or manual creation of a digital model of the selected orthodontic appliance can be carried out. The creation of a digital model can take place for all selected orthodontic appliances at this time. Furthermore, it is conceivable for the creation of a digital model to take place after the end and before the start of each individual treatment step, as long as the datasets necessary therefor are stored, or are stored in a patient file. If, at any time during the entire duration of an orthodontic treatment, unforeseen developments occur, i.e. the teeth do not change their position as intended, it is possible to capture the tooth regions to be correspondingly treated again during the entire orthodontic treatment (step 40) and to create another digital 3D model relating to the captured tooth regions (step 41). It is furthermore possible to prepare additional digital images of the tooth region to be treated orthodontically during the ongoing treatment, so that corrections and/or amendments to the orthodontic appliances/aids can be carried out. It is advantageous to store the digital images since the course of treatment is documented.

Optionally, after the creation of a digital model of the selected orthodontic appliance, a skeletonization process 51 can be carried out. With the aid of a skeletonization process, the digital model of the selected orthodontic appliance can be digitally reduced to a minimum with regard to excess material. This results in less consumption of material. Furthermore, the wearing feel or wearing comfort and thus acceptance by the patient are improved.

The preparation of a digital dataset 52 of the digital model of the selected and optionally skeletonized orthodontic appliance follows.

The control of a rapid prototyping printer subsequently takes place on the basis of the provided digital dataset, so that the plastics material constituents of the orthodontic appliance can be printed (step 53).

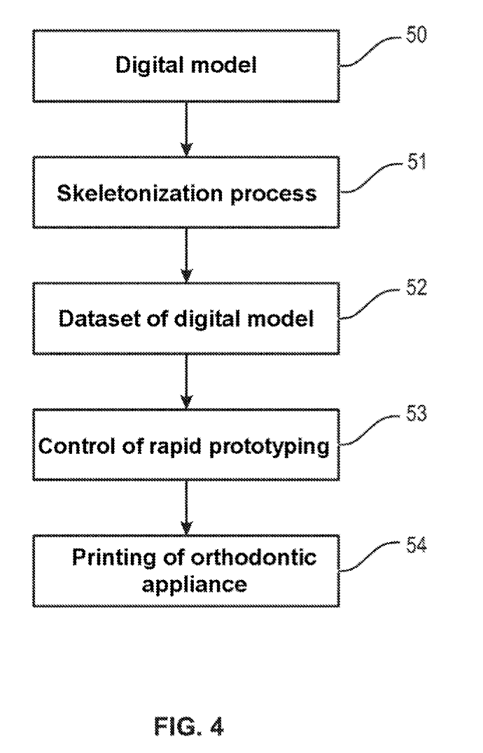

If for example an aligner is produced, the method according to the invention ends with step 54. If further aligners are intended to be produced, steps 50 to 54 according to FIG. 4 are repeated with each individual aligner to be produced.

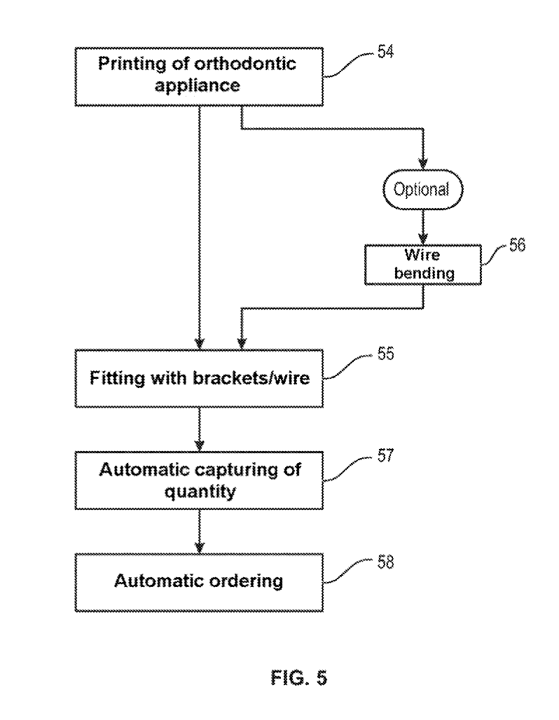

According to FIG. 5, an expanded exemplary embodiment of the method according to the invention is illustrated, insofar as a device for indirect sticking, i.e. a tray, is intended to be produced. After the plastics material constituents of the orthodontic appliance have been printed 54, automatic and/or manual fitting 55 of the printed orthodontic appliance with at least one bracket and/or at least one bent wire takes place. The fitting can be carried out with the aid of a robot system, present in the rapid prototyping printer, having gripping arms. To this end, the rapid prototyping printer has a plurality of chambers with brackets and/or wires and/or other aids present therein. Preferably, a plurality of brackets of different embodiments and/or different sizes and/or from different manufacturers are stored in individual chambers. If a wire is intended to be applied to the orthodontic appliance together with the brackets, the optional method step 56, namely the bending of a wire, has to be carried out. To this end, the rapid prototyping printer again comprises a corresponding wire bending device. The dimensions of the wires and the wire sequences can be calculated in an appropriate manner and stored. Individual tagging of the work yet to be carried out is also possible, it being possible in this connection to make an assignment to patient data which are stored for example in digital patient files.

It should be noted that the fitting can also be carried out manually, i.e. the dentist or dental technician can attach or apply individual brackets or all of the brackets to the positions of the orthodontic appliance that are intended therefor.

In a further embodiment of the invention, the rapid prototyping printer can be connected to an automatic inventory control system, so that automatic capturing of the quantity of brackets and/or wire and/or aids, for example screws and/or used plastics material, present in chambers of a rapid prototyping printer can take place, so that it is possible to pass the captured data on to an inventory control system (step 57). The individual elements are identified for example with the aid of RFID labels and/or QR codes.

In the context of the automated inventory control system, it is possible to create automated ordering operations relating to required brackets and/or required wire and/or required plastics material.

The user can read corresponding statistics relating to the capacity and productivity of the rapid prototyping printer at any time. At the same time, conclusions about the further materials that are still necessary and are required for treatment can be drawn via the used materials, so that orders can be adapted in a corresponding manner. By way of a further interface with the various settlement programs, corresponding settlement items and texts can be proposed to the operator, so that in the case of a settlement, to be accordingly made, with regard to the produced orthodontic appliance, further sources of error can be minimized.

It should be noted that the present method, the present device and the arrangement are suitable both for producing trays for attaching the brackets to the outer side of the teeth and for producing trays for attaching brackets to the inner side of the teeth.

The device for producing any orthodontic appliance, especially a rapid prototyping printer, can be located both in a practice and in the laboratory, the data for producing the orthodontic appliances also being able to be passed on to the rapid prototyping machine by remote access or via an Internet connection. The completed orthodontic appliances can already be packaged in the printer by a corresponding packaging unit, thereby making hygienic shipping or hygienic storage of the orthodontic appliances possible.

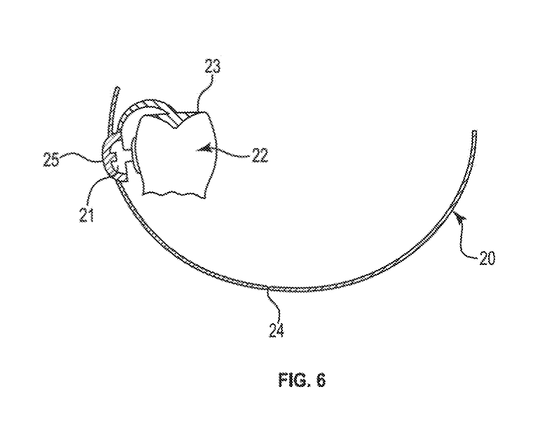

FIG. 6 shows a device according to the invention for the indirect sticking of orthodontic appliances. What is referred to as a tray 20, which consists of plastics material constituents, is illustrated. A bracket 21, for example, is intended to be attached to a tooth 22, so that the tray 20 has a bracket 21 at this position assigned to the tooth 22. For example, an adhesive can be present on that surface of the bracket that faces the tooth 22, in order to connect the bracket to the tooth. In the region of the bracket 21, the plastics material of the tray is formed with an increased material thickness, in order to establish a sufficient hold of the bracket 21 in the tray 20. This thickened region can also be referred to as a cap 25.

Furthermore, the plastics material can be provided in the regions of the occlusal tooth surface 23 and the incisal edge. As a result, exact positioning of the tray 20 or of the bracket 21 on the tooth 22 is established, inter alia. Occlusal means directed towards the chewing surface. Occlusal also means the contact between the teeth in the upper jaw and the teeth in the lower jaw.

Moreover, the plastics material of the tray can be provided with a predetermined breaking point 24, so that, after the brackets have been stuck or attached to the teeth, the removal of the plastics materials can be simplified. The predetermined breaking point 24 has the effect that the tray 20 can be removed from the oral cavity of the patient 1 in a plurality of individual parts. Finally, it should be noted that materials other than plastics material can also be produced in the 3D printer unit. These can be for example ceramics or alloys.

### LIST OF REFERENCE SIGNS

1 Patient

2 Intraoral scanner

3 Data processing unit

4 Line

5 Rapid prototyping printer

6 Screen

7 Keyboard

8 Mouse

9 Planning unit

10 Cover

11 Holder

12 Gripping arm

13 Storage container

14 Storage chamber

20 Tray

21 Bracket

22 Tooth

23 Occlusal tooth surface

24 Predetermined breaking point

25 Cap

40-58 Method steps

1. An arrangement for producing an orthodontic appliance and/or orthodontic, the arrangement comprising a device for digitally capturing a tooth region to be treated orthodontically, a data processing unit and at least one 3D printer, the data processing unit controlling the at least one 3D printer and being connected or connectable to the device for digitally capturing a tooth region to be treated orthodontically, wherein the orthodontic appliance and/or orthodontic aid is required for the entire duration of an orthodontic treatment.

2. The arrangement according to claim 1, characterized in that the orthodontic appliance is a device for the indirect sticking of a fixed orthodontic appliance.

3. The arrangement according to claim 1, characterized in that the device for digitally capturing a tooth region to be treated orthodontically comprises one or more of an intraoral scanner, an x-ray device, a computed tomograph, or a digital volume tomograph.

4. The arrangement according to claim 1, characterized in that the data processing unit is connected or connectable to a data input device.

5. A device for producing an orthodontic appliance, the device comprising at least one 3D printer unit comprising at least one chamber for filling with brackets, wherein the orthodontic appliance is required for the entire duration of an orthodontic treatment.

6. The device according to claim 5, characterized in that the 3D printer unit comprises at least one chamber for filling with wire.

7. The device according to claim 5 further comprising an automated inventory control system having a monitoring device for checking the filling level of at least one chamber for filling with brackets and/or of the at least one chamber filled with wire and/or of the at least one chamber filled with aids and/or of the plastics material to be processed.

8. The device according to claim 5, characterized in that the 3D printer unit comprises a robotic system having at least one gripping device.

9. The device according to claim 5, characterized in that the 3D printer unit comprises at least one wire bending device.

10. A device according to claim 5, characterized in that the 3D printer unit comprises a UV-light resistant cover.

11. The device according to claim 5, characterized in that the 3D printer unit comprises a packaging device.

12. A method for producing an orthodontic appliance, the method comprising:
capturing a tooth region to be treated orthodontically;
preparing a digital three-dimensional model of the tooth region to be treated orthodontically;
automatically and/or manually creating a modified tooth arrangement;
subdividing positional changes of teeth that are to be carried out into individual orthodontic treatment steps to be carried out in succession;
preparing a dataset with regard to an individual orthodontic treatment step to be carried out; and
linking the individual orthodontic treatment step to be carried out with an orthodontic appliance from a selection of a plurality of orthodontic appliances,
wherein the orthodontic appliance is required for the entire duration of an orthodontic treatment.

13. The method according to claim 12, further comprising automatically and/or manually creating a digital model of the orthodontic appliance.

14. The method according to claim 12 further comprising digitally reducing the orthodontic appliance to a minimum with regard to excess material using a skeletonization process.

15. The method according to claim 12, further comprising providing a digital dataset of a digital model of the orthodontic appliance.

16. The method according to claim 15, further comprising controlling a 3D printer using the digital dataset to print one or more material constituents of the orthodontic appliance, the material constituents comprising one or more of plastics, alloys, and ceramics.

17. The method according to claim 16, further comprising automatically and/or manually fitting the printed orthodontic appliance with at least one bracket and/or at least one bent wire.

18. The method according to claim 16, further comprising automatically capturing a quantity of one or more of brackets, wire, plastics material, and auxiliary elements disposed within one or more chambers of the 3D printer; and transmitting the one or more captured quantities to an inventory control system.

19. The method according to claim 18, further comprising generating one or more automated ordering operations based on the quantity of the one or more of the brackets, the wire, the plastics material, and the auxiliary elements.

20. A device for the indirect sticking of an orthodontic appliance, characterized in that the orthodontic appliance has at least one predetermined breaking point.

21. A device according to claim 20, characterized in that the orthodontic appliance is a skeletonized orthodontic appliance and/or a skeletonized orthodontic aid.
